(19) United States
(12) Plant Patent Application Publication
(10) Pub. No.: US 2006/0075536 P1
(43) Pub. Date: Apr. 6, 2006

(54) Schefflera 'Fan'

(50) SCHEFFLERA ARBORICOLA; SCHEFFLERA

(76) Inventors: Eric T. Anderson, Escondido, CA (US); Peter Pittman, Homestead, FL (US)

Correspondence Address: DAVID B. WALLER & ASSOCIATES, 5677 OBERLIN DRIVE, SUITE 214, SAN DIEGO, CA, 92121, US

(21) Appl. No.: 10/958,912

(22) Filed: Oct. 5, 2004

(51) Int. Cl. A01H 5/00

(52) U.S. Cl. PLT/377

(57) ABSTRACT

A distinct cultivar of Schefflera plant named ‘Fan’, characterized by its upright plant habit: rapid growth rate; closely-spaced leaves and parted palmate leaves that are palmately veined, very glossy and dark green which give a dense full appearance.

### TECHNICAL FIELD

The present invention is a new and distinct cultivar of Schefflera plant, botanically known as Schefflera arboricola, and herein referred to by the name ‘Fan’.

### BACKGROUND OF THE INVENTION

The new cultivar was discovered by the inventors in a controlled environment in Homestead Fla. United States in September 2003 as a naturally occurring mutation of Schefflera arboricola. The new cultivar was observed as a single plant in a group of plants of Schefflera arboricola. Compared to plants of Schefflera arboricola, the plants of the new Schefflera are denser having closely-spaced and parted palmate leaves that are palmately veined, very glossy and dark green.

Asexual propagation of the new cultivar by terminal cuttings taken in the controlled environment in Elfin Forest, Calif., United States, has shown that the unique features of this new Schefflera are stable and reproduced true to type in successive generations.

The new Schefflera has not been observed under all possible environmental conditions. The phenotype may vary somewhat with variations in environment such as temperature and light intensity, without, however, any variance in genotype. The following traits have been repeatedly observed and are determined to be the unique characteristics of plants of the new Schefflera. These characteristics in combination distinguish the new Schefflera as a new and distinct cultivar:

1. Upright plant habit.

2. Rapid growth rate.

3. Parted palmate leaves that are palmately veined and closely spaced.

4. Very glossy and dark green leaves.

### BRIEF DESCRIPTION OF THE PHOTOGRAPHS

The accompanying color photographs illustrate the overall appearance of the new Schefflera, showing the colors as true as it is reasonably possible to obtain in color reproductions of this type.

FIG. 1 is a photograph side perspective view of a typical potted plant of the new Schefflera.

FIG. 2 is a close up photograph of a typical leaf of the new Schefflera plant. Leaf colors in the photograph may differ from the actual colors due to light reflectance.

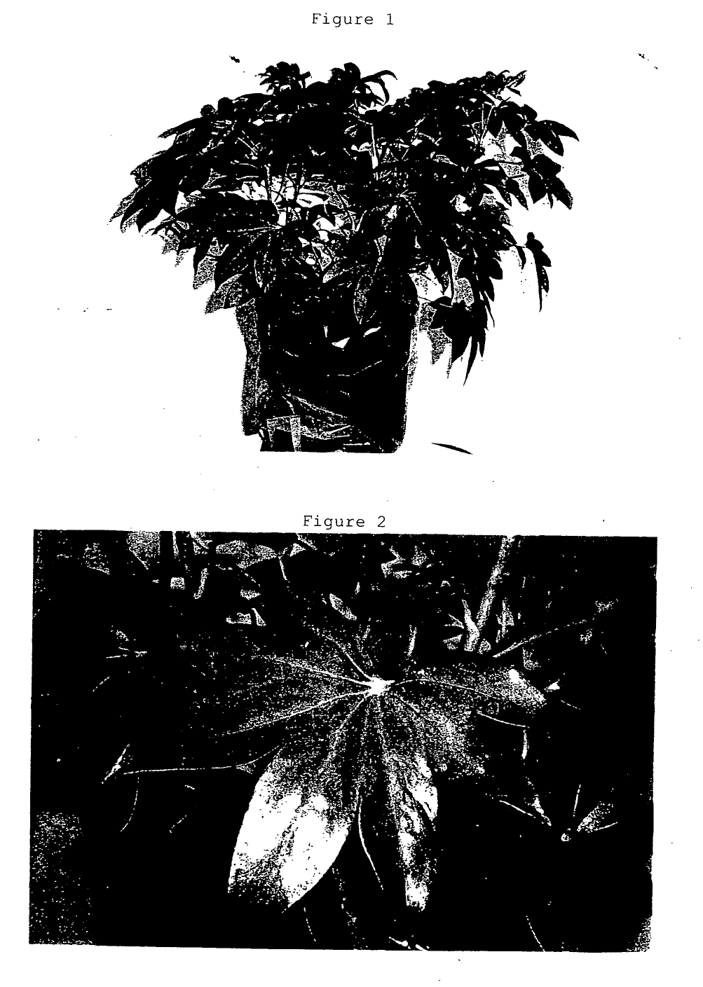

### DETAILED BOTANICAL DESCRIPTION

In the following description, color references are made to The Royal Horticultural Society Colour Chart except where general terms of ordinary dictionary significance are used. The following observations and measurements describe four month old plants in 25 cm diameter containers grown in Elfin Forest, Calif., USA under commercial practice in a translucent plastic-covered greenhouse with day and night temperatures averaging 80° F. and 64° F. respectively. Average light level was 1,500 foot candles.
Botanical classification: Schefflera arboricola cultivar ‘Fan’.
Parentage: naturally occurring mutation of Schefflera arboricola.
Propagation type: by terminal cuttings.
Time to initiate roots: Winter: about 28 days at 70° F., Summer: about 21 days at 76° F.
Rooting habit: numerous, fleshy and thick.
Plant description:
Plant shape: Mostly erect and upright, inverted triangle, symmetrical.
Growth habit: bushy, dense and full.
Plant type: tropical evergreen tree.
Plant height: about 300 to 400 cm from soil level to top of leaf plane.
Plant diameter: about 100 cm.
Plant vigor/crop time: high rapid growth rate under optimum temperature. Appropriate for 10 to 25 cm and larger containers. About 20 weeks are required to produce 100 cm tall plants.
Stem description/shape: nearly round with internode length 2.5 to 4.5 cm. Color: R.H.S. 144A.
Petioles: Average length about 15 cm, color R.H.S. 144B
Foliage description: Leafs: Shape: parted palmate having on average from 5 to 8 tips per leaf and are palmately veined.
Diameter: about 11 to 16 cm. Texture: leathery, thick, smooth, very glossy, glabrous, and durable. Color: upper surface: glossy, dark green, R.H.S. 139A, Lower surface: R.H.S. 146A. Venation: R.H.S. 145A.
Flower description: Flowers have not been observed on plants of the new Schefflera.
Disease/insect resistance: This Schefflera has no special disease resistance as compared with other Scheffleras observed to date on plants grown under commercial shadehouse and greenhouse conditions.

It is claimed:

1. A new and distinct cultivar of Schefflera plant named ‘Fan’, as illustrated and described.
